(19) United States
(12) Patent Application Publication
(10) Pub. No.: US 2018/0221523 A1
(43) Pub. Date: Aug. 9, 2018

(54) Smoke Scent

(71) Applicant: Jorge E. Mallo, JR., Miami Shores, FL (US)

(72) Inventor: Jorge E. Mallo, JR., Miami Shores, FL (US)

(21) Appl. No.: 15/863,890

(22) Filed: Jan. 6, 2018

Related U.S. Application Data
(60) Provisional application No. 62/443,602, filed on Jan. 6, 2017.

(51) Int. Cl. A61L 2/16

(52) U.S. Cl. CPC A61L 2/16

(57) ABSTRACT

A smoke scented spray, cream or other applicator used to mask uniquely human scents while hunting. Natural smoke is collected and incorporated with other optional ingredients. The hunter applies the formulation to their clothes or body to improve avoiding animal detection and increasing chances of successful hunting.

Related U.S. Patent Documents

| Application Number | Filing Date | Pat. No. |
|---|---|---|
| 62/443,602 | Jan. 6, 2017 | |

With the above and other related objects in view, the invention consists in the details of construction and combination of parts as will be more fully understood from the following description, when read in conjunction with the accompanying drawings in which:

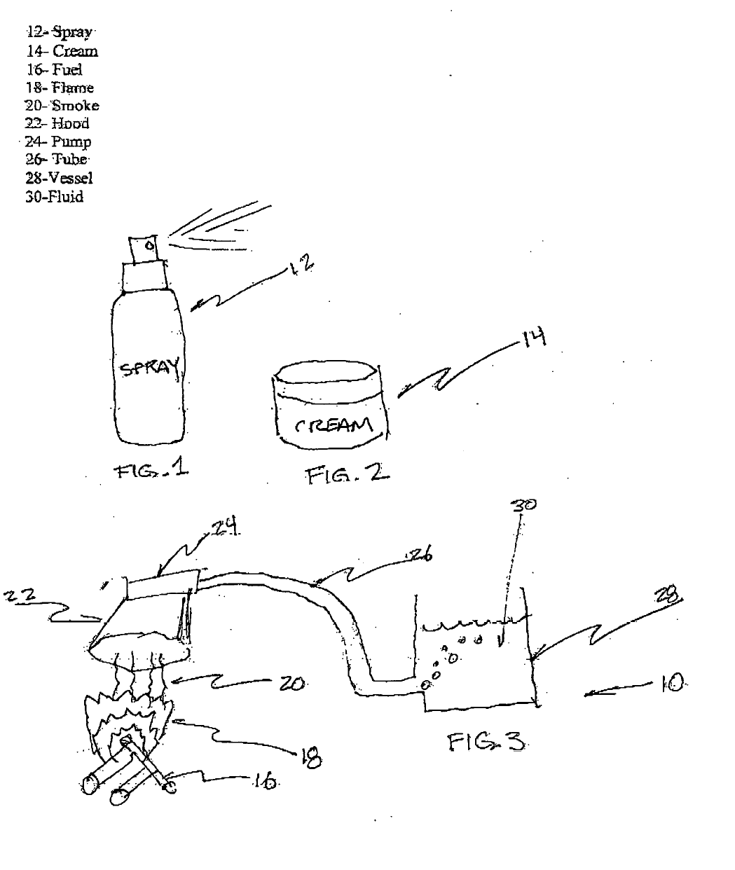

FIG. 1 shows a perspective view of an example of a spray applicator for possible use with a smoke scent.

FIG. 2 shows a perspective view of an example of a cream format for potential use with a smoke scent.

FIG. 3 shows a perspective view of an example of a smoke collection apparatus.

While this invention may be embodied in many different forms, there are described in detail herein specific embodiments of the invention. This description is an exemplary of the principles of the invention and is not intended to limit the invention to the particular embodiments illustrated and described.

For the purpose of this disclosure, like reference 1 numerals in the figures shall refer to like features unless otherwise indicated or is obvious by context.

The subject device, method of manufacture and use is sometimes referred to as the device, the invention, the scent, the smoke scent, the mask, machine or other similar terms. These terms may be used interchangeably as context requires and from use the intent becomes apparent. The masculine can sometimes refer to the feminine and neuter and vice versa. The plural may include the singular and singular the plural as appropriate from a fair and reasonable interpretation in the situation.

Referring now to the drawings, where the present invention is generally referred to with numeral 10, it can be observed that it basically includes a spray 12, a cream 14, a fuel 16, a flame 18, smoke 20, a hood 22, a pump 24, a tube 26, a vessel 28 and a fluid 30. Some of the above elements are contextual, optional, exemplary and provided for a fuller understanding or may not be required in all embodiments of the inventive concept. In at least one version of the invention the smoke scent is carried in a liquid, colloidal, crème or gel. As the viscosity of the completed smoke scent compound is affected by its constituent ingredients, the preferred delivery method may be different.

For example, a solution of less than about twenty 1 five centipoises could be effectively sprayed with a device such as the spray 12 depicted in FIG. 1. Thicker viscosities up to about fifty-thousand centipoises could be dispensed in a tube or bottle (not shown in the figures). Thicker formulations yet, up to about several million centipoises may be dispensed from a container such as the cream 14 container depicted by example in FIG. 2. These viscosity values are merely exemplary and may be higher or lower depending on the nature of the compound, the specific application and application method.

The process for manufacturing a smoke scent begins with collecting natural smoke and related combustion particulates. The smoke and possibly the collection medium (eg. Where fluid 30 is water) is then combined with optional ingredients such as emulsifiers, carriers, stickers, preservatives, scents, colorants, dyes, insecticides and sun screens resulting in a homogenous product with a smoke scent along with characteristics of the amendments.

During typical use, the smoky combination is then applied to the skin or clothes of the hunter prior to the hunt or even during the hunt. The smoky scent can mask scents carried on a human hunter that animals can detect. Animal detection of scents not found in nature, such as human body odors, personal hygiene products, gun oil, plastic and other odors that may be associated with humans can alert animals to the presence of the human hunter. An alerted animal may flee or be an higher alert making hunting and stalking quarry more difficult.

By use of the smoke scent animals have a difficult time specifically identifying human sourced scents and therefore will not be alerted to the presence of the hunter. Methods and tactics will also have an effect on hunting, as has always been the case. However, every advantage a hunter has over her prey increases the probability that she will be able to have a successful hunt and provide food for her family.

FIG. 3 shows an example of a method of collecting natural smoke particulate matter. A fuel 16 is ignited to support a flame 18. The burning fire converts some of the fuel 16 into smoke 20. Instead of exhausting the smoke 20 or allowing it to dissipate in the air, the smoke 20 is collected. Collection efficiency can be improved by the use of a hood 22. The ascending heat from the flame 18 can carry the smoke 20 up into a hood 22. Attached to the hood 22 is an air pump 24 that accepts the smoke 20 and pumps it through a tube 26 into a lower part of a vessel 28 containing a fluid 30. As the smoke 20 is bubbled through the liquid 30 a portion of the smoke 20 is dissolved into the liquid 30. This is one example of how smoke 20 is collected and other effective methods may optionally be employed to make the smoke scent.

Optionally, near where the tube 26 empties into the 1 vessel 28 there may be included a diffuser to break up the incoming smoke 20 and air into smaller bubbles thus having a greater total surface area of bubbles being introduced into the fluid 30 thereby increasing the efficiency of dissolving the smoke 30 into the fluid 30.

The fluid 30 can be effective when comprised of a liquid, such as water. Various mineral, vegetable and synthetic oils may be used in the alternative or in combination with water. Similarly, alcohols such as ethanol, isopropyl alcohols or others may be the primary solvent comprising the fluid 30 or may merely be adjuncts to other primary ingredients.

Any of the oils or alcohols may also alternatively be added to the formulation after the step of collecting the smoke, as opposed be being a part of the fluid 30. Recipes can be adjusted based on the user's preference, hunting type, concentrations of ingredients and effectiveness.

Other ingredients that are neither alcohols or oils may also increase effectiveness of the completed formulation. For example, emulsifiers, carriers, stickers, preservatives, scents, colorants, dyes, insecticides and sun screens may bring added benefits to the hunter than simply a smoke scent alone would provide.

For example, many hunters will forego insect repellants because of the associated smells of the insecticide and other ingredient. However, masked with smoke, insecticides are less detectable by animals. The smoke scent itself also provides some degree of insect deterrence that any hunter would appreciate.

The smoke scent formulation can be mixed or amended with human scent neutralizers, eliminators or maskers such as are used in some fresh earth formulas that are commercially available. Smoke scent concentrate can be added to other products that the hunter wants to use or wear to symbiotically help the hunter to seamlessly and undetectably mesh into their environment.

Animals of a particular region may be more inclined to ignore smoke from burning material from their natural environment. If an animal senses smoke of a type they are familiar with then they may ignore the source if detected in concentrations less than a wildfire. For example, hogs in mid-Florida are accustomed to grass fires and sugar cane controlled burns. They are likely to not be overly aggravated by such a familiar smell and thus not be put on high alert.

Different fuels 16 may be used to alter the character of the ultimate product. For example, conifer fuels such as fir or pine may more closely match the woods available in the northwest. Aspen or birch may be preferred because of natural prevalence in the northern Midwest and Canada. Even grasses, leaves, crop waste and other organic 1 matter may be used for a nuanced smoke scent.

The foregoing description conveys the best understanding of the objectives and advantages of the present invention. Different embodiments may be made of the inventive concept of this invention.

It is to be understood that all matter disclosed herein is to be interpreted as illustrative, and not in a limiting sense.

What is claimed is:

1. A method of masking uniquely human odors through the use of a smoke scented spray, cream or other applicator.

2. The method of claim 1, wherein the smoke scent would be dispersed as a liquid spray.

3. The method of claim 1, wherein the smoke scent would be incorporated into or dispersed as a cream or gel.

4. The method of claim 1, wherein the smoke scent would be part of a method of preparing for hunting comprising at least one of: a) applying a liquid spray or a powder, each containing smoke scent to at least a portion of a hunter's body prior to or during hunting; b) washing a portion of the hunter's body with one of a liquid soap or a bar soap, moistened pre-packaged wash cloth, hair conditioner, shampoo, dry shampoo containing smoke scent; c) Brushing with a toothpaste, or rinsing the mouth of the hunter with a mouthwash each containing smoke scent; d) applying a deodorant or antiperspirant containing smoke scent or e) a foot powder containing smoke scent.

5. The method of claim 1, wherein the smoke scent would be part of preparing for hunting comprising: washing hunting apparel in a liquid or powered laundry detergent and dryer sheet containing smoke scent.

6. The method of claim 1, wherein the smoke scent would be dispersed with a powdered carrier such as corn starch, sodium bicarbonate, talk or other powdered carrier for the purposes of detecting wind direction.

7. The method of claim 1, wherein the smoke scent would be incorporated/added to a lip balm, skin cream conditioner/moisturizer or a camouflage face paint.

8. The method of claim 1, wherein the smoke scent would be incorporated/added to a gun or bow lubricant such as a grease, oil or wax.

9. The method of claim 1, wherein the smoke scent would be incorporated/added to a powdered carrier or a liquid concentrate for reconstitution with water later to save weight on a flight or when backpack hunting in remote areas.

10. The method of claim 1, wherein the smoke scent would be incorporated/added to other plant oils, game (animal) bodily fluid or scent eliminators.

11. The method of claim 1, wherein the smoke scent would be incorporated/added to hand sanitizer.

12. The method of claim 1, wherein the smoke scent would be incorporated/added to insect repellent or sunscreen.

13. The method of claim 1, wherein the smoke scent would be incorporated/added to an ultraviolet (UV) light glow reducing chemical, that can reduce the amount of UV light that is reflected off of hunt clothing. Deer and Turkey are particularly sensitive to the reflection of UV light off of clothing.
